(19) United States
(12) Patent Application Publication
(10) Pub. No.: US 2015/0202131 A1
(43) Pub. Date: Jul. 23, 2015

(54) HIGH PROTECTION WATER-RESISTANT SUNSCREEN COMPOSITION

(71) Applicant: Riemann Trading ApS, Hillerød (DK)

(72) Inventor: Lene Britze, Kokkedal (DK)

(73) Assignee: Riemann Trading ApS, Hillerød (DK)

(21) Appl. No.: 14/416,152

(22) PCT Filed: Jul. 25, 2013
(86) PCT No.: PCT/DK2013/050251

§ 371 Date: Jan. 21, 2015

(30) Foreign Application Priority Data
Jul. 26, 2012 (DK) PA 2012 00477

(51) Int. Cl. A61K 8/37; A61K 8/81; A61K 8/34; A61Q 17/04

(52) U.S. Cl. CPC A61K 8/37; A61Q 17/04; A61K 8/8176; A61K 8/8152; A61K 8/8158; A61K 8/34; A61K 2800/48

(57) ABSTRACT

Thus, one aspect of the invention relates to a sunscreen composition comprising at least one sunscreen agent, at least one film forming polymer, and at least one solvent, wherein said film forming polymer is present in an amount of less than 2.0% (w/w). Another aspect of the present invention relates to a process for the manufacture of a sunscreen composition comprising the steps of i) providing at least one solvent, at least one film forming polymer in an amount of less than 2.0% (w/w), and at least one sunscreen agent, ii) admixing the components of step i) to provide a sunscreen composition.

### TECHNICAL FIELD OF THE INVENTION

The present invention relates to sunscreen compositions. In particular the present invention relates to sunscreen compositions with reduced amounts of film forming polymers and high water resistance.

### BACKGROUND OF THE INVENTION

It is a well-established fact that UV irradiation causes skin burns, loss of skin elasticity and the appearance of wrinkles, promoting premature skin aging. In addition, UV irradiation promotes the triggering of the erythemal reaction or accentuates this reaction in certain individuals and may even be the source of phototoxic or photoallergic reactions. Last, but not least important, UV irradiation has carcinogenic effects. For these reasons, continuous need exists for means for protecting the skin against the harmful effects of UV irradiation.

Numerous requirements exist for dermatological compositions for photo-protection of the human skin: Compositions are preferred which have a pleasant feel and are formulated as non-greasy sprays, milks and creams that are easily applied. It is further preferred that the compositions exhibit non-adherence to sand, as users of these compositions are often in contact with sand at beaches and play areas.

In addition to being convenient for the user the compositions must contain adequate solvent to hold the necessary amount of UV-filter. Need continues to exist for compositions which simultaneously have a low or zero content of preservatives, while exhibiting an optimum stability which is maintained throughout the time of storage and ultimate use of the products. Finally, in instances where a particularly high and lasting protection is desired, as for children's skin or for sensitive skin, it is also preferable to employ compositions which exhibit good water resistance and which thus retain a very good photo protective power even after coming into contact with water. For example WO 2009/016537 describes sunscreen formulations comprising dimethicone derivatives, wherein from about 75% to about 100% of an SPF rating of the composition is retained after immersion into water for 80 minutes. Corresponding formulation without dimethicone derivatives perform poorly with respect to water resistance.

Many photoprotective, dermatological compositions are oil-in-water emulsions. However, one of the disadvantages of traditional oil-in-water emulsions is that they very easily lose their effectiveness with respect to UV protection as soon as they come into contact with water; this because the screening agents which they contain in their aqueous phase are removed by the water, during bathing in the sea or in swimming pools, for example, or, alternatively, under the shower or when playing water sports, and the overall photoprotective power of these compositions thus is greatly reduced. Thus, sunscreen compositions which are solvent solutions with a low water content rather than oil-in-water emulsions would be advantageous.

Generally, film forming polymers or water proofing agents are added to sunscreen compositions to enhance resistance to water in vivo. For example WO 2011/100275 describes a sunscreen composition formulated as a spray comprising a film former in an amount of 2.5% by weight (w/w). Film forming polymers are however expensive additives and generally it is desirable to reduce the amount of additives in cosmetic compositions such as sunscreen agents.

Therefore, improved or alternative sunscreen compositions which have a high SPF rating and simultaneously maintain a high water resistance, are non-greasy and easily applicable, while comprising reduced amount of additives, such as film forming polymers would be advantageous.

### SUMMARY OF THE INVENTION

Thus, an object of the present invention relates to the provision of improved or alternative sunscreen compositions.

In particular, it is an object of the present invention to provide a sunscreen composition that solves the above mentioned problems of providing sunscreen compositions with reduced amount of additives, particularly film forming polymers, while maintaining a very high level of water resistance in vivo and a high SPF rating.

Thus, one aspect of the invention relates to a sunscreen composition comprising at least one sunscreen agent, at least one film forming polymer, and at least one solvent, wherein said film forming polymer is present in an amount of less than 2.0%(w/w).

Another aspect of the present invention relates to a process for the manufacture of a sunscreen composition comprising the steps of
i) providing at least one solvent, at least one film forming polymer in an amount of less than 2.0%(w/w), and at least one sunscreen agent,
ii) admixing the components of step i) to provide a sunscreen composition.

The present invention will now be described in more detail in the following.

### DETAILED DESCRIPTION OF THE INVENTION

### Definitions

Prior to discussing the present invention in further details, the following terms and conventions will first be defined:

In the present context “sunscreen composition” or photoprotective composition is to be understood as a liquid composition comprising agents that reduces the amount of UV radiation being absorbed by the skin, when said sunscreen composition is applied to the skin. A sunscreen composition may either absorb or block the UV rays. UV radiation may include UVA and UVB radiation. Sunscreen compositions may be formulated as solutions, emulsions, milks or oils, and may be applied to the skin by the means of sprays, creams, or lotions.

In the context of the present invention “sunscreen agent” is any cosmetologically approved agent capable of adsorbing or blocking UV radiation, such as UVA and/or UVB radiation. Sunscreen agents that absorb UV radiation are also referred to as UV filters, UVA, UVB or broad spectrum filters. Such sunscreen agents are often organic compounds or molecules, which absorb light in the UV region, the UV region being at wavelengths from 290 to 400 nanometers. Sunscreen agents that block the UV rays may include substances that leave a white coating on the skin, such as certain metal oxides including for example titanium oxide or other white substances.

In the present context “film forming polymer”, also sometimes referred to as a “waterproofing agent”, is a polymer capable of forming a film on e.g. skin as the carrier or solvent that it is dissolved in evaporates. A film forming polymer may be a uniform homo-polymer or a co-polymer, such as for example a block-copolymer. The polymer should be suitable for use in cosmetic products and may preferably be cosmetologically approved. In the present context the term “co-polymer” relates to a polymer formed when two (or more) different types of monomer are linked in the same polymer chain, as opposed to a homo-polymer where only one monomer is used.

Herein “solvent”, also sometimes referred to as a carrier, is any liquid capable of dissolving the components of the sunscreen composition. A solvent may be a mixture of different solvents or it may be a single solvent. The solvent should be suitable for use in cosmetics and may preferably be cosmetologically approved.

In relation to the present invention the term “alcohol” refers to an organic compound in which a hydroxyl group (—OH) is attached to a saturated carbon atom. Alcohols have the general formula ROH, where R may be a lower-aliphatic or cyclic carbon chain and may include aromatic rings. Alcohols are further classified as primary, secondary or tertiary, according to the position of the hydroxyl group on the lower-aliphatic carbon chain. Alcohols may also be classified by the number of hydroxyl groups present in the molecule; for example, a compound with two hydroxyl groups (a diol), and with three hydroxyl groups (a triol). The alcohol may comprise a denaturant. In the present context the term “Alcohol Denaturant” relates to ethyl alcohol that is denatured with one or more denaturing agents. One such denaturing agent may be Denatonium Benzoate.

A “solution” according to the present invention consists of a solvent and a range of ingredients which are all entirely dissolved in said solvent. Thus, a solution does not comprise any solid dispersed material, i.e. it is not a dispersion. Also a solution does not comprise other liquid immiscible components which may or may not form vesicles that are dispersed in said solution, i.e. the solution is not an emulsion.

In the present context an “anhydrous” composition means a composition which is essentially free of water. Essentially free of water entails that no water is added to a composition, however minor amounts of water may be present since other components added to the composition may comprise small amount of water. Preferably, anhydrous composition means a composition entirely free of water.

In the present context the term “minimal erythema dose (MED)” refers to the quantity of erythema-effective energy (expressed as Joules per square meter or milli joules per square centimetre) required to produce the first perceptible, redness reaction in the skin with clearly defined borders.

Guidelines for labelling of Sun Protection Factor (SPF) number and product categories are suggested by COLIPA (The European Cosmetic Toiletry and perfumery Association) in Europe. These are listed in the table below:

| Labeled Category | Labeled SPF | Measured SPF |
|---|---|---|
| Low Protection | 6 | 6.0-9.9 |
| | 10 | 10.0-14.9 |
| Medium Protection | 15 | 15.0-19.9 |
| | 20 | 20.0-24.9 |
| | 25 | 25.0-29.9 |
| High Protection | 30 | 30.0-49.9 |
| | 50 | 50.0-59.9 |
| Very high protection | 50+ | ≧60 |

In the present context the term “Sun protection factor” (SPF) refers to the UV energy required to produce an MED on protected skin divided by the UV energy required to produce an MED on unprotected skin. The “sun protection factor” term may also be defined as the ratio of the minimum erythemal dose on protected skin (MEDₚ) to the minimum erythemal dose on unprotected skin (MEDᵤ):

SPF=MEDₚ/MEDᵤ

The Sun Protection Factor value on an individual subject (SPF₁), for any product or composition, either before or after water immersion, may be determined as the ratio of the minimum erythemal dose on protected skin (MEDₚ) to the minimum erythemal dose on unprotected skin (MEDᵤ) of the same subject.

Further, the term “static sun protection factor”, (SPFₛ), relates to the sun protection factor before water immersion, while the term “wet sun protection factor” (SPF_{w}) refers to the sun protection factor after water immersion.

The static and wet SPF values are determined according to the current published

International Sun Protection Factor (SPF) Test Method (I-SPF-TM) as defined in 2006 by COLIPA (CTFA SA-JCIA-CFTA US) as well as international standard ISO 24444:2010(E).

Herein “thickening agent” is any agent which is capable of increasing the viscosity of a cosmetic composition such as for example a sunscreen composition. The agent may preferably be cosmetologically approved.

Sunscreen Composition

A first aspect of the present invention is a sunscreen composition comprising at least one sunscreen agent, at least one film forming polymer, and at least one solvent, wherein said film forming polymer is present in an amount of less than 2.0%(w/w).

The sunscreen composition may preferably be a sprayable sunscreen composition. Herein “sprayable” or “sprayable sunscreen composition” relates to a composition which is capable of being formulated and dispensed as a spray. In the context of sunscreen compositions a further preferred quality to a sprayable composition is that said composition when dispensed using a spray dispenser, preferably a spray dispenser not comprising a propellant, is dispensed in a way which allows sufficient and even distribution of the composition onto the skin. In this respect a sprayable sunscreen composition should preferably have a viscosity which allows it to form an aerosol upon dispensing it as a spray, and it should preferably be dispensable in a volume which allows for sufficient coverage of the skin. Preferably the present sunscreen composition is formulated as a spray.

In order to enhance the sunscreen compositions performance as a sprayable sunscreen composition the viscosity of said sunscreen composition may be below 120 cP at 25° C., such as below 110 cP at 25° C., 100 cP at 25° C., 80 cP at 25° C., 60 cP at 25° C., 40 cP at 25° C., 20 cP at 25° C., 15 cP at 25° C., such as below 10 cP at 25° C. The sunscreen composition may thus have a viscosity in the range of 1-120 cP at 25° C., such as in the range of 1-110 cP at 25° C., 1-100 cP at 25° C., 1-80 cP at 25° C., 1-60 cP at 25° C., 1-40 cP at 25° C., 1-30 cP at 25° C., 1-20 cP at 25° C., 1-15 cP at 25° C., 1-14 cP at 25° C., 2-13 cP at 25° C., 3-12 cP at 25° C., preferably in the range of 4-11 cP at 25° C.

The present sunscreen composition comprises a film forming polymer, which provides for better water resistance of the composition. In a preferred embodiment the film forming polymer is selected from the group consisting of polyvinylpyrrolidone (PVP), acrylates, acrylamides, and any copolymers thereof or any combination thereof. Preferably the film forming polymer is selected from the group consisting of acrylate/octylacrylamide copolymer, acrylates copolymer, acrylates/C₁₂₋₂₂ alkyl methacrylate copolymer, butylated PVP), VA/butyl maleate/isobornyl acrylate copolymer vinyl caprolactam/VP/dimethylaminoethyl methacrylate copolymer, most preferably acrylate/octylacrylamide copolymer.

The inventors surprisingly found that the film forming polymer would provide excellent water resistance of the present sunscreen compositions even at very low weight percentages. Thus, the film forming polymer may preferably be present in the sunscreen composition in an amount of less than 1.8%(w/w), such as less than 1.6%(w/w), less than 1.4%(w/w), less than 1.2%(w/w), less than 1.1%(w/w), less than 1.0%(w/w), less than 0.9%(w/w), less than 0.8%(w/w), such as less than 0.76%(w/w). In another embodiment the film forming polymer is present in an amount in the range of 0.1-2.0%(w/w), such as in the range of 0.2-2.0%(w/w), e.g. in the range of 0.3-2.0%(w/w), such as in the range of 0.3-2.0%(w/w), e.g. in the range of 0.4-2.0%(w/w), such as in the range of 0.4-2.0%, e.g. in the range of 0.5-1.5%(w/w), such as in the range of 0.5-1.2%(w/w), e.g. in the range of 0.6-1.0%(w/w), such as in the range of 0.6-0.9%(w/w), e.g. in the range of 0.7-0.8%(w/w), such as about 0.75%(w/w).

Unexpectedly, water does not need to be added to the present sunscreen composition, since solvation of the added components and the desired properties of the composition, such as stability, viscosity and clarity is achieved without adding water. Thus in as preferred embodiment said sunscreen composition comprises water in an amount of 0-2%(w/w), such as 0-1%(w/w), such as 0-0.8%(w/w), 0-0.6%(w/w), 0-0.4%(w/w), 0-0.2%(w/w), 0-0.1%(w/w), 0-0.05%(w/w), such as 0-0.01%(w/w). In an even more preferred embodiment the composition is essentially anhydrous and most preferably the composition is anhydrous.

The solvent of the present invention may preferably be an alcohol. Said alcohol may be a mono alcohol having 2-8 carbon atoms, such as 2-4 carbon atoms. Preferably the alcohol is selected from the group consisting of ethanol, 1-propanol, 2-propanol, ethyleneglycol, 1-butanol, 2-butanol or any combination thereof. Ethanol is particularly preferred. The alcohol may comprise a denaturant, particularly when the alcohol is ethanol. The solvent content may preferably be in the range of 10-90%(w/w), such as in the range of 15-85%(w/w), 20-80%(w/w), 25-70%(w/w), 28-65%(w/w), 30-60%(w/w), 30-50%(w/w), such as in the range of 30-40%(w/w) or in the range of 20-30%(w/w).

Preferably the sunscreen composition according to the present invention is a solution. Thus the components of the present compositions are preferably dissolved entirely in the solvent without forming for example emulsions or milks, and even more preferably the sunscreen composition does not comprise emulsifiers.

The sunscreen composition comprises one or more sunscreen agents, which provide the adsorption or blocking of the UV radiation, before it reaches the skin. The sunscreen agent may be selected from the group consisting of Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide, Benzophenone-3, Ethylhexyl Methoxycinnamate, Octocrylene, Butyl Methoxydibenzoylmethane (BMBM), Diethylamino hydroxybenzoyl Hexyl Benzoate, Diethylhexyl Butamido Triazone, PABA, camphor benzalkonium methosulfate, phenylbenzimidazole sulfonic acid, terephthalidene dicamphor sulfonic acid, benzylidene camphor sulfonic acid, polyacrylamidomethyl benzylidene camphor, PEG-25 PABA, isoamyl p-methoxycinnamate, ethylhexyl triazone, drometrizole trielloxane, 4-methylbenzylidene camphor, 3-benzylidene camphor, ethylhexyl salicylate, ethylhexyl dimethyl PABA, benzophenone-4, methylene bis-benztriazolyl tetramethylbutylphenol, disodium phenyl dibenzimidazole tetrasulfonate, bis-ethylhexyloxyphenol methoxyphenol triazine, methylene bisbenzotriazolyl tetramethylbutylphenol, bisethylhexyloxyphenol methoxyphenyl triazine and any combination thereof.

In an even more preferred embodiment the at least one sunscreen agent is selected from the group consisting of Homosalate, Octocrylene, Ethylhexyl Methoxycinnamate, Butyl Methoxydibenzoylmethane (BMBM), Diethylamino Hydroxybenzoyl Hexyl Benzoate, diethylhexyl butamido triazone, ethylhexyl salicylate or any combination thereof. Even more preferred is a composition comprising Homosalate, Octocrylene, Ethylhexyl Methoxycinnamate, Butyl Methoxydibenzoylmethane (BMBM), Diethylamino Hydroxybenzoyl Hexyl Benzoate and diethylhexyl butamido triazone.

The amount of each individual sunscreen agent used in a given sunscreen composition is determined by the desired SPF value and regulatory limitations for the percentage (w/w) of sunscreen agent allowed in sunscreen compositions for a given jurisdiction. To achieve broad range UV protection it may advantageous to add both UVA, UVB and broad range UV filters. Finally, if the desired SPF is not achieved with a given set of sunscreen agents, further amounts of a different sunscreen agent may be added or preferably the SPF may be boosted by other means, such as described herein. Therefore, in preferred embodiments Diethylamino Hydroxybenzoyl Hexyl Benzoate is used in concentrations of at the most 10%(w/w), e.g. in the range of 0.1-10%(w/w), such as in the range of 3-10%(w/w), e.g. in the range 5-10%(w/w), such as in the range of 7-10%(w/w), and/or Ethylhexyl Methoxycinnamate is used in concentrations of at the most 2%(w/w), e.g. in the range of 0.01-2%(w/w), such as in the range of 0.01-1%(w/w), e.g. in the range 0.01-0.05%(w/w) and/or Octocrylene is used in concentrations of at the most 10%(w/w), e.g. in the range of 0.1-10%(w/w), such as in the range of 0.5-8%(w/w), e.g. in the range 1-6%(w/w), such as in the range of 3-5%(w/w), e.g. in the range 6-10%(w/w), such as in the range of 8-10%(w/w) and/or Butyl Methoxydibenzoylmethane (BMBM) is used in concentrations of at the most 5%(w/w), such as in the range of 0.5-5%(w/w), e.g. in the range 3-5%(w/w), such as in the range of 4-5%(w/w) and/or homosalate is used in concentrations of at the most 10%(w/w), e.g. in the range of 0.1-10%(w/w), such as in the range of 0.5-8%(w/w), e.g. in the range 6-10%(w/w), such as in the range of 8-10%(w/w) and/or diethylhexyl butamido triazone is used in concentrations of at the most 5%(w/w), such as in the range of 0.1-5%(w/w), e.g. 1-5%(w/w), such as 3-5%(w/w), and/or ethylhexyl salicylate is used in concentrations of at the most 5%(w/w), such as in the range of 0.1-5%(w/w), e.g. 1-5%(w/w), such as 3-5%(w/w).

In a highly preferred embodiment Diethylamino Hydroxybenzoyl Hexyl Benzoate is used in concentrations about 10%(w/w), and optionally Ethylhexyl Methoxycinnamate is used in concentrations of 0.01 (w/w), and Octocrylene is used in concentrations of about 10%(w/w), and Butyl Methoxydibenzoylmethane (BMBM) is used in concentrations of about 5%(w/w) and homosalate is used in concentrations of about 10%(w/w), and diethylhexyl butamido triazone is used in concentrations of about 4.5%(w/w), and ethylhexyl salicylate is used in concentrations of about 5%(w/w).

The sunscreen composition may comprise several sunscreen agents to achieve the desired SPF value. Thus in a preferred embodiment the composition comprises at least 2 sunscreen agents, such as 3 sunscreen agents, e.g. 4 sunscreen agents, such as 5 sunscreen agents, e.g. 6 sunscreen agents, such as 7 sunscreen agents.

In a preferred embodiment the present composition comprises butyloctyl salicylate. Said salicylate may be present in the composition in an amount in the range of 0.1-15%(w/w), such as 0.1-10%(w/w), 0.5-10%(w/w), 1.0-10%(w/w), 2-10%(w/w), 3-10%(w/w), 4-10%(w/w), 5-10%(w/w), 6-10%(w/w), 7-10%(w/w), 8-10%(w/w), 9-10%(w/w), 9.5-10%(w/w), such as about 10%(w/w). Butyloctyl salicylate may function as SPF booster and as co-solvent.

Silicone derivatives are used as emollients in for example sunscreen compositions and they also provide for improved skin feel. Since silicone derivatives may become subject of regulatory limitations “silicone alternatives” are also available, which provide similar effects to silicone derivatives but are mostly non-silicone compounds or compound mixtures derived from natural sources. Thus, in a preferred embodiment the composition further comprises a siloxane or a silane or silicone alternative, such as a siloxane, silane or silicone alternative selected from the group consisting of C₁₃₋₁₆ isoparaffin, C₁₂₋₁₄ isoparaffin, C₁₃₋₁₅ Alkane, Isodecane and Hydrogenated Polydecene and Bis-Behenyl/Isostearyl/Phytostearyl Dimer Dilinoleyl/Dimer Dilinoleate, Silyburn Marianum Ethyl Ester, C₈₋₁₈ Alkyl Ester, cyclic dimethyl polysiloxane or cyclopentasiloxane. The siloxane, silane or silicone alternative may be present in an amount in the range of 0.1-20%(w/w), such as in the range of 0.5-15%(w/w), e.g. in the range of 1-10%(w/w), such as in the range of 2-7%(w/w), e.g. in the range of 2-5%(w/w), such as in the range of 2-4%(w/w), e.g. in the range of 2-3%(w/w).

The present sunscreen composition may advantageously comprise an alkoxylated alcohol, which may serve as an emollient and/or co-solvent. The alkoxylated alcohol may preferably a long chain C₁₁-C₁₅ aliphatic alkoxylated alcohol and may be selected from the group consisting of PPG-11 stearyl ether, PPG-15 Stearyl ether, or any combination thereof. The alkoxylated alcohol may be added in an amount in the range of 0.1-20%(w/w), such as in the range of 0.5-15%(w/w), e.g. in the range of 1-10%(w/w), such as in the range of 2-7%(w/w), e.g. in the range of 5-7%(w/w).

The present composition may further comprise an ester, which may serve as an emollient and/or co-solvent. The ester may preferably be selected from the group consisting of Ethylhexyl Benzoate, Dibutyl Adipate, Neopentyl Glycol Dicaprylate/Dicaprate, C₁₂₋₁₅ Alkyl benzoate or any combination hereof. The ester may be present in an amount which is less 15%(w/w) such as less than 10%(w/w) such as less than than 5%(w/w), such as less than 4%(w/w), less than 3%(w/w), less than 2%(w/w), less than 1%(w/w), less than 0.5%(w/w), less than 0.2%(w/w), such as less than 0.1%(w/w).

The composition may comprise a thickening agent. Said thickening agent may preferably be a polysaccharide thickening agent. It may preferably be a cellulosic material, and may preferably be selected from the group consisting of cellulose, carboxymethyl hydroxyethylcellulose, hydroxypropyl methylcellulose, methyl hydroxyethylcellulose, microcrystalline cellulose, sodium cellulose sulphate, hydroxyethylcellulose, hydroxypropylcellulose, and any mixtures thereof.

The ability of a sunscreen composition to be water resistant is highly desirable as explained above. A remarkably high water resistance was achieved for the present sunscreen composition, even when using very small amounts of film forming polymer as waterproofing agent. Thus, in a preferred embodiment the composition having a static sun protection factor after the composition has been applied to the skin of a human, the wet sun protection factor of the composition is at least 90% of the static sun protection factor, such as at least 95%, 100%, 105%, 110%, 115%, such as at least 120%, when the composition is contacted with water, particularly after the composition has been immersed in water in vivo for 80 minutes.

In a highly preferred embodiment the composition having a static sun protection factor after the composition has been applied to the skin of a human, the sun protection factor of the composition is capable of being increased to a higher wet sun protection factor when the composition is contacted with water, particularly after the composition has been immersed in water in vivo for 80 minutes. A preferred sunscreen composition is a composition wherein the wet sun protection factor is increased by at least 5% relative to the static sun protection factor, such as increased by at least 10% relative to the static sun protection factor, e.g. increased by at least 15% relative to the static sun protection factor, such as increased by at least 20% relative to the static sun protection factor.

The water resistance of a sunscreen composition may be measured according to internationally or regionally approved standards such as for example the Guidelines for Evaluating Sun Product Water Resistance (COLIPA 2005). A sunscreen having a particularly high water resistance according to the regional or international standard may be designated by a “very water resistance” (VWR) value. Very Water Resistance is measured in vivo after a test subject has been immersed in water for a total of 80 minutes i.e. 4 times 20 minutes with 15 min drying time in between. Thus, in a preferred embodiment the present invention is a composition wherein the Very Water Resistance value of said composition is above 50, such as above 60, above 70, above 80, such as above 85, above 90, above 95, above 100, above 105, such as above 110.

A highly preferred embodiment of the present invention is to provide a sunscreen composition with a high SPF value. Therefore in a preferred embodiment the present invention relates to a sunscreen composition wherein the sunscreen protection factor of said composition is at least 40, such as at least 42, at least 44, at least 46, at least 48, at least 50, at least 51, at least 52, at least 53, at least 54, such as at least 55. It sunscreen composition is preferably provided that achieves a high SPF value and a high water resistance while using a minimal amount of sunscreen agents. Thus, in preferred embodiment the composition comprises no more than 7 sunscreen agents and wherein the sun protection factor is at least 30, such as at least 32, at least 34, at least 36, at least 38, at least 40, at least 42, at least 44, at least 46, at least 48, at least 50, at least 52, at least 53, at least 54, such as at least 55, such as no more than 6 sunscreen agents and wherein the sun protection factor is at least 30, such as at least 32, at least 34, at least 36, at least 38, at least 40, at least 42, at least 44, at least 46, at least 48, at least 50, at least 52, at least 53, at least 54, such as at least 55.

It is desirable to provide a composition which delivers a high protection against the particularly harmful UVA radiation. UVA protection may either be expressed via the so-called COLIPA ratio or via the Boots star rating system, the guidelines for both of these rating systems being described in the below examples. Thus, the present sunscreen compositions may preferably have a COLIPA ratio below 3, such as below 2, below 1.8. Preferably the present sunscreen composition has a boots star rating of at least 4 stars.

The overall stability and particularly the photo-stability of sunscreen compositions must preferably be high in order to provide prolonged protection against UV radiation without the need for excessive and repeated topical administration during exposure to the sun. Therefore a preferred embodiment of the present invention is a sunscreen composition wherein the SPF value of said composition 10 hours after application in vivo is at least 60%, such as at least 65%, at least 70%, at least 75%, such as at least 78% of the SPF value of said sunscreen composition 15 minutes after application in vivo. Preferably said SPF value is the static SPF value.

### Method of Manufacturing a Sunscreen Composition

Another aspect of the present invention is a process for the manufacture of a sunscreen composition comprising the steps of
i) providing at least one solvent, at least one film forming polymer in an amount of less than 2.0%(w/w), and at least one sunscreen agent,
ii) admixing the components of step i) to provide a sunscreen composition.

Generally heating is used when manufacturing sunscreen compositions, however for the present invention the inventors surprisingly found that heating was unnecessary, and all the components dissolves to a satisfactory level without applying heat, beyond the heat generated during solvation and stirring. Therefore, preferably the mixture or composition is not heated during the process and the temperature is in the range of 0-60° C., such as in the range of 5-55° C., 15-50° C., 20-50° C., 30-45° C., such as about 40° C. Preferably, no water is added to the composition during said process, and even more preferably the components provided in step i) are admixed in the order provided in step i). Further additives as described herein may be added in step i). Each component added is dissolved under stirring before the next component is added. Stirring time may vary from 1-25 minutes.

All the embodiments described for the aspect relating to a sunscreen composition may naturally also apply to the sunscreen composition described for the present method of manufacturing.

It should be noted that embodiments and features described in the context of one of the aspects of the present invention also apply to the other aspects of the invention. All patent and non-patent references cited in the present application, are hereby incorporated by reference in their entirety.

The invention will now be described in further details in the following non-limiting examples.

### EXAMPLES

### Example 1

### Sunscreen Compositions and Manufacture Thereof

Sunscreen compositions were prepared according to the tables shown below for compositions A and B.

TABLE 1
Sunscreen composition A (reduced film former)

| | | Amount |
|---|---|---|
| Steps | Ingredients | w/w % |
| 1.1 | Alcohol denat. (solvent) | 35.74 |
| 1.2 | Acrylates/Octylacrylamide Copolymer (Film former, | 0.75 |
| | a synthetic polymer) | |
| 1.3 | PPG-15 Stearyl Ether (Emolient/weak emulisifier, | 6.0 |
| | an alkoxylated alcohol) | |
| 1.4 | Butyloctyl Salicylate (Emolient) | 10.0 |
| 1.5 | Cyclopentasiloxane (Emolient, a Siloxane/silane) | 3.0 |
| 1.6 | Ethylhexyl Methoxycinnamate (Sunscreen agent, | 0.01 |
| | an ester) | |
| 1.7 | Octocrylene (Sun screen agent, an ester) | 10.0 |
| 1.8 | Homosalate (Sun Screen Agent) | 10.0 |
| 1.9 | Ethylhexyl Salicylate (Sun Screen Agent) | 5.0 |
| 1.10 | Diethylhexyl Butamido Triazone (Sun Screen Agent) | 4.5 |
| 1.11 | Butyl methoxydibenzoylmethane (Sun Screen Agent) | 5.0 |
| 1.12 | Diethylamino Hydroxybenzoyl Hexyl Benzoate (Sun | 10.0 |
| | screen agent) | |
| | Total | 100 |

TABLE 2
Sunscreen composition B (reduced film former)

| | | Amount |
|---|---|---|
| Steps | Ingredients | w/w % |
| 1.1 | Alcohol denat. (solvent) | 23.74 |
| 1.2 | Acrylates/Octylacrylamide Copolymer (Film former, | 0.75 |
| | a synthetic polymer) | |
| 1.3 | PPG-15 Stearyl Ether (Emolient/weak emulisifier, | 6.0 |
| | an alkoxylated alcohol) | |
| 1.4 | C12-15 Alkyl Benzoate (Emolient) | 21.5 |
| 1.5 | Cyclopentasiloxane (Emolient, a Siloxane/silane) | 3.0 |
| 1.6 | Ethylhexyl Methoxycinnamate (Sunscreen agent, | 0.01 |
| | an ester) | |
| 1.7 | Octocrylene (Sun screen agent, an ester) | 10.0 |
| 1.8 | Homosalate (Sun Screen Agent) | 10.0 |
| 1.9 | Ethylhexyl Salicylate (Sun Screen Agent) | 5.0 |
| 1.10 | Diethylhexyl Butamido Triazone (Sun Screen Agent) | 5.0 |
| 1.11 | Butyl methoxydibenzoylmethane (Sun Screen Agent) | 5.0 |
| 1.12 | Diethylamino Hydroxybenzoyl Hexyl Benzoate (Sun | 10.0 |
| | screen agent) | |
| | Total | 100 |

The compositions were manufactured by addition of the listed components in the order they are listed under stirring using a Silverson L4RT high shear mixer. Stirring time in between additions of components is variable between 1-25 minutes. No heating is of the solution was performed. The temperature of the mixture was between 20° C. and 40° C. at all times.

### Example 2

### Water Resistance of Sunscreen Compositions

The effect of reduced amounts of film forming polymer on the water resistance of the compositions of the present invention was measured in vivo. Sunscreen compositions A and B were prepared according to example 1 and subjected to in vivo SPF determination as prescribed by the Guidelines for Evaluating Sun Product Water Resistance (COLIPA 2005). The tests were performed by Institute Dr. Schrader Hautphysiologie, Holzminden, Germany.

As described the wet SPF as measured in vivo is the SPF value measured after application of sunscreen and subsequent immersion of the test individual in water for 80 minutes. The immersion in water for 80 minutes is divided into 4 immersions of 20 minutes with 15 minutes of drying time in air before first immersion, in between immersions, and after the fourth immersion.

The very water resistance value (in percentage) is calculated as:

VWR=100×SPF_{w}/SPFₛ

where the lower limits of the confidence intervals (95% CI) is used for SPF_{w} and SPFₛ based on statistics performed on the data from 12 test subjects.

TABLE 3
in vivo measurements of SPF water resistance
for sunscreen compositions A and B

| | A | B |
|---|---|---|
| Amount of film forming polymer %(w/w) | 0.75 | 0.75 |
| Very Water Resistance* | >100% | >90% |

*Based on SPF (static and wet) measurements in vivo on 11 and 6 individuals respectively

The tests show that the compositions of the present invention retain a very high water resistance even with very low levels of film forming polymer present, which justifies the label “very water resistant” according to the recommendations in the abovementioned guidelines. The requirement for this label is a water resistance of 50% or more.

### Example 3

### UVA Protection and Boots Star Rating of Sunscreen Compositions

The protection against UVA radiation may be determined by the COLIPA ratio as defined in the COLIPA guideline (in vitro method for the determination of the UVA protection factor and “critical wavelength” values of sunscreen products”. Guideline, COLIPA, March 2011). The COLIPA ratio is calculated as

COLIPA Ratio=SPF_{label}/UVA-PF

where UVA-PF is the actual “SPF” obtained for UVA irradiation measured in vitro according to the guidelines. The present compositions A and B were tested and displayed a COLIPA ratio of 1.6 and 1.7 respectively, which fulfils the recommendation from COLIPA which is a ratio below 3.

The UVA protection was also determined by the Boots star rating system (Measurement of UVA:UVB ratios according to the boots star rating system [2001 revision]; the Boots company PLC, Nottingham NG2 3AA, UK, September 2011), which is based on the ratio between UVA and UVB protection before and after exposing the sample to UV irradiation. The best Boots star rating of five stars (*****) requires a UVA:UVB ratio above 0.9 before UV exposure and above 0.86 after UV exposure. The second best rating of four stars (****) requires a UVA:UVB ratio above 0.8 before UV exposure and above 0.76 after UV exposure. Compositions A and B fulfils these criteria.

TABLE 6
Boots star rating of composition A

| | Before UV exposure | | | | | After UV exposure | | | | |
|---|---|---|---|---|---|---|---|---|---|---|
| | Integral of | | Area per unit | | | Integral of | | Area per unit | | |
| | UV range | | wavelength | | | UV range | | wavelength | | |
| Plate* | UVB | UVA | UVB | UVA | Ratio | UVB | UVA | UVB | UVA | Ratio |
| 1 | 63.9 | 145.0 | 2.13 | 1.81 | 0.85 | 63.7 | 143.0 | 2.12 | 1.79 | 0.84 |
| 2 | 61.4 | 137.7 | 2.04 | 1.72 | 0.84 | 61.1 | 136.7 | 2.04 | 1.71 | 0.84 |
| 3 | 63.2 | 143.1 | 2.11 | 1.79 | 0.85 | 62.3 | 140.6 | 2.08 | 1.76 | 0.85 |
| 4 | 62.1 | 139.4 | 2.07 | 1.74 | 0.84 | 62.0 | 137.5 | 2.07 | 1.72 | 0.83 |
| No. | 4 | 4 | 4 | 4 | 4 | 4 | 4 | 4 | 4 | 4 |
| Mean | 62.6 | 141.3 | 2.09 | 1.77 | 0.85 | 62.3 | 139.4 | 2.08 | 1.74 | 0.84 |
| Std. dev. | 1.1 | 3.4 | 0.04 | 0.04 | 0.005 | 1.1 | 2.9 | 0.04 | 0.04 | 0.006 |

| | | |
|---|---|---|
| λ_{c} [nm] | 382 | 382 |

Star rating: ****
*mean product quantity = 0.78 mg/cm₂, mean plate temp. = 31.6° C.

TABLE 7
Boots star rating of composition B

| | Before UV exposure | | | | | After UV exposure | | | | |
|---|---|---|---|---|---|---|---|---|---|---|
| | Integral of | | Area per unit | | | Integral of | | Area per unit | | |
| | UV range | | wavelength | | | UV range | | wavelength | | |
| Plate* | UVB | UVA | UVB | UVA | Ratio | UVB | UVA | UVB | UVA | Ratio |
| 1 | 77.3 | 165.2 | 2.58 | 2.07 | 0.80 | 77.2 | 161.2 | 2.57 | 2.01 | 0.78 |
| 2 | 81.7 | 174.5 | 2.72 | 2.18 | 0.80 | 80.6 | 169.7 | 2.69 | 2.12 | 0.79 |
| 3 | 81.6 | 172.7 | 2.72 | 2.16 | 0.79 | 81.1 | 170.9 | 2.70 | 2.14 | 0.79 |
| 4 | 80.1 | 170.1 | 2.67 | 2.13 | 0.80 | 79.1 | 167.4 | 2.64 | 2.09 | 0.79 |
| No. | 4 | 4 | 4 | 4 | 4 | 4 | 4 | 4 | 4 | 4 |
| Mean | 80.2 | 170.6 | 2.67 | 2.13 | 0.80 | 79.5 | 167.3 | 2.65 | 2.09 | 0.79 |
| Std. dev. | 2.1 | 4.0 | 0.07 | 0.05 | 0.004 | 1.7 | 4.3 | 0.06 | 0.05 | 0.005 |

| | | |
|---|---|---|
| λ_{c} [nm] | 380 | 380 |

Star rating: ****
*mean product quantity = 0.93 mg/cm₂, mean plate temp. = 22.7° C.

### Example 4

### Viscosity of Sunscreen Compositions

The viscosity of the present sunscreen compositions must be within certain limits for the compositions to remain sprayable. Thus, the viscosity of composition A was investigated along with compositions having more thickening agent (solvent amounts being adjusted accordingly). The viscosities were measured at 25° C. at 60 rpm using a Brookfield LVDV-II+P viscometer with a small sample adapter and Spindle S18.

TABLE 11
Viscosity of composition A and variants

| Composition | Amount HPC (%(w/w)) | Viscosity (cP) |
|---|---|---|
| A | 0 | 8 |
| A1 | 0.03 | 10 |
| A2 | 0.10 | 14 |
| A3 | 0.20 | 19 |

HPC = hydroxypropylcellulose

The tests show that at 0 and 0.03%(w/w) HPC (composition A and A1) the viscosity remains is ideal, whereas at higher levels of thickening agent the viscosity reaches levels which may start to affect the sprayability, particularly when applying standard retail type topical administration pumps.

### REFERENCES

WO 2009/016537
WO 2011/100275
Commission Recommendation of 22 Sep. 2006 on the efficacy of sunscreen products and the claims made relating thereto (notified under document number C(2006) 4089) (Text with EEA relevance) (2006/647/EC); Official Journal of the European Union, L 265/3

1. A sunscreen composition comprising:
at least one sunscreen agent,
at least one film forming polymer, and
at least one solvent, wherein said film forming polymer is present in an amount of less than 2.0%(w/w).

2-44. (canceled)

45. The sunscreen composition according to claim 1, wherein said composition is a sprayable sunscreen composition.

46. The sunscreen composition according to claim 1, wherein said film forming polymer is selected from the group consisting of polyvinylpyrrolidone (PVP), acrylates, acrylamides, and any copolymers thereof or any combination thereof.

47. The sunscreen composition according to claim 1, wherein said film forming polymer is present in an amount of less than: 1.8%(w/w), 1.6%(w/w), 1.4%(w/w), 1.2%(w/w), 1.1%(w/w), 1.0%(w/w), 0.9%(w/w), 0.8%(w/w), or 0.76%(w/w).

48. The sunscreen composition according to claim 1, wherein the sunscreen protection factor of said composition is at least: 40, 42, 44, 46, 48, 50, 51, 52, 53, 54, or 55.

49. The sunscreen composition according to claim 1, wherein said composition is anhydrous.

50. The sunscreen composition according to claim 1, wherein said solvent is an alcohol.

51. The sunscreen composition according to claim 1, wherein said composition comprises butyloctyl salicylate.

52. The sunscreen composition according to claim 51, wherein said butyloctyl salicylate is present in the composition in an amount in the range of: 0.1-15%(w/w), 0.1-10%(w/w), 0.5-10%(w/w), 1.0-10%(w/w), 2-10%(w/w), 3-10%(w/w), 4-10%(w/w), 5-10%(w/w), 6-10%(w/w), 7-10%(w/w), 8-10%(w/w), 9-10%(w/w), or 9.5-10%(w/w), or in an amount of about 10%(w/w).

53. The sunscreen composition according to claim 1, wherein the composition further comprises a siloxane, silane, a silicone alternative, siloxane, silane, C₁₃₋₁₆ isoparaffin, C₁₂₋₁₄ isoparaffin, C₁₃₋₁₅ Alkane, Isodecane, Hydrogenated Polydecene, Bis-Behenyl/Isostearyl/Phytostearyl Dimer, Dilinoleyl/Dimer, Dilinoleate, Silyburn Marianum Ethyl Ester, C₈₋₁₈ Alkyl Ester, cyclic dimethyl polysiloxane or cyclopentasiloxane.

54. The sunscreen composition according to claim 1, wherein said composition comprises a thickening agent.

55. The sunscreen composition according to claim 54, wherein said thickening agent is a polysaccharide thickening agent.

56. The sunscreen composition according to claim 55, wherein said polysaccharide thickening agent is selected from the group consisting of cellulose, carboxymethyl hydroxyethylcellulose, hydroxypropyl methylcellulose, methyl hydroxyethylcellulose, microcrystalline cellulose, sodium cellulose sulphate, hydroxyethylcellulose, and hydroxypropylcellulose, or any mixtures thereof.

57. The sunscreen composition according to claim 1, wherein the viscosity of said sunscreen composition is below: 120 cP at 25° C., 110 cP at 25° C., 100 cP at 25° C., 80 cP at 25° C., 60 cP at 25° C., 40 cP at 25° C., 20 cP at 25° C., 15 cP at 25° C., or 10 cP at 25° C.

58. The sunscreen composition according to claim 1, wherein the composition comprises at least 2 sunscreen agents, 3 sunscreen agents, 4 sunscreen agents, 5 sunscreen agents, 6 sunscreen agents, or 7 sunscreen agents.

59. The sunscreen composition according to claim 1, wherein the composition having a static sun protection factor after the composition has been applied to the skin of a human, the wet sun protection factor of the composition is at least 90%, 95%, 100%, 105%, 110%, 115%, or 120% of the static sun protection factor, when the composition is contacted with water.

60. The sunscreen composition according to claim 1, wherein the composition having a static sun protection factor after the composition has been applied to the skin of a human, the sun protection factor of the composition is capable of being increased to a higher wet sun protection factor when the composition is contacted with water.

61. The sunscreen composition according to claim 1, wherein the wet sun protection factor is increased by at least 5% relative to the static sun protection factor, increased by at least 10% relative to the static sun protection factor, increased by at least 15% relative to the static sun protection factor, or increased by at least 20% relative to the static sun protection factor.

62. The sunscreen composition according to claim 1, wherein the Very Water Resistance value of said composition is above 50, 60, 70, 80, 85, 90, 95, 100, 105, or 110.

63. A process for the manufacture of a sunscreen composition comprising:
i) providing at least one solvent, at least one film forming polymer in an amount of less than 2.0% (w/w), and at least one sunscreen agent, and
ii) admixing the components of step i) to provide a sunscreen composition.

64. A process according to claim 63, wherein the mixture is not heated during the process and the temperature is in the range of 0-60° C.
